(19) United States
(12) Patent Application Publication
(10) Pub. No.: US 2006/0084814 A1
(43) Pub. Date: Apr. 20, 2006

(54) Process for the preparation of zonisamide

(76) Inventors: Bobba Venkata Siva Kumar, Navi Mumbai (IN); Shekhar Bhaskar Bhirud, Navi Mumbai (IN); Batchu Chandrasekhar, Navi Mumbai (IN); Sanjay Anantha Kale, Navi Mumbai (IN)

Correspondence Address: M. CARMEN & ASSOCIATES, PLLC, 170 OLD COUNTRY ROAD, SUITE 400, MINEOLA, NY, 11501, US

(21) Appl. No.: 11/133,543

(22) Filed: May 20, 2005

Related U.S. Application Data
(60) Provisional application No. 60/572,979, filed on May 20, 2004.

(51) Int. Cl. C07D 261/20

(52) U.S. Cl. 548/241

(57) ABSTRACT

The present invention provides an improved process for the preparation of zonisamide or a derivative thereof comprising (a) reacting 1,2-benzisoxazole-3-methane-sulfonic acid with a halogenating agent in a first organic solvent to provide benzisoxazole methane sulfonyl halide; and, (b) reacting benzisoxazole methane sulfonyl halide with an amine in a second organic solvent to form zonisamide or a derivative thereof.

### CROSS-REFERENCE TO RELATED APPLICATION

This application claims the benefit under 35 U.S.C. §119 to Provisional Application No. 60/572,979, filed May 20, 2004 and entitled “PROCESS FOR THE PREPARATION OF ZONISAMIDE,” the contents of which are incorporated by reference herein.

### BACKGROUND OF THE INVENTION

1. Technical Field

The present invention relates generally to processes for the preparation of zonisamide and derivatives thereof.

2. Description of the Related Art

The present invention relates to a process for the preparation of zonisamide, which is known as 1,2-benzisoxazole-3-methane sulfonamide, of Formula I: 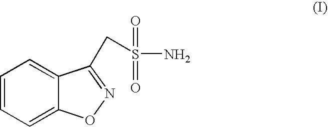
Zonisamide is an anticonvulsant drug used in the treatment of epilepsy, and is sold under the brand name Zonegran®. See, e.g., The Merck Index, Thirteenth Edition, p. 1817 (2001), monograph 10244 and Physician's Desk Reference, “Zonegran,” 58th Edition, pp. 1231-1235 (2004).

U.S. Pat. No. 4,172,896 discloses a process for preparing zonisamide as generally depicted in Scheme I below:

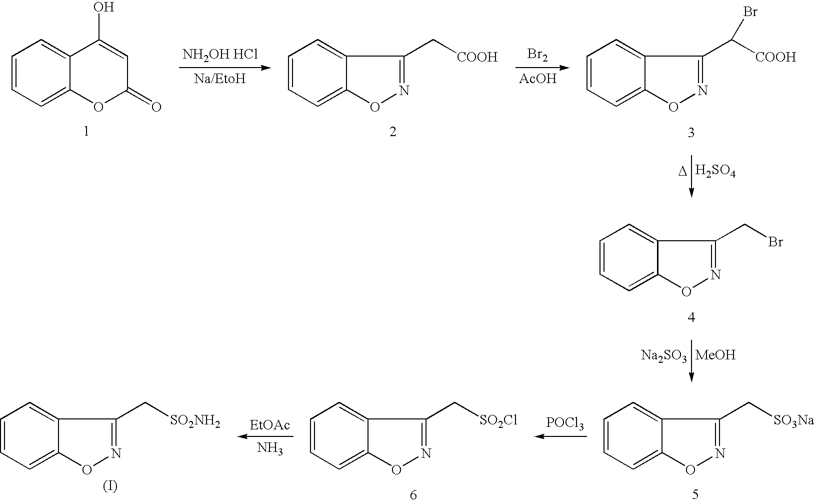

In Scheme I, hydroxycoumarin (1) was reacted with hydroxyl amine hydrochloride under Posner reaction conditions to give 1,2-benzisoxazole-3-acetic acid (“BOA”) (2). BOA (2) was brominated through the unstable bromo acid (3) to provide zonisamide-bromide (4). Zonisamide-bromide (4) was converted to 1,2-benzisoxazole-3-methane sulfonic acid sodium salt (“BOS—Na”) (5) by reaction with sodium sulfite. BOS—Na (5) was converted in two steps into the zonisamide (I) by first reacting BOS—Na with a halogenating agent to form benzisoxazole methane sulfonyl chloride (“BOS—Cl”) (6) which is then further reacted with ammonia in ethyl acetate to produce zonisamide (I).

Problems associated with this process include the use of sodium metal in the preparation of BOA which (1) is flammable and results in the reaction being unsafe on a commercial scale and (2) forms BOA and the side-reaction product O-hydroxy-acetophenone-oxime to the extent of about 30%. The high percentage of the side reaction products as well as the difficulty of using the aforementioned process on an industrial scale due to the use of a sodium metal render this process unfavorable, and thus the need for an improved process for preparing zonisamide remains.

U.S. Pat. No. 6,677,458 discloses a process for the preparation of BOS—Na salt by sulfonating BOA with chlorosulfonic acid and dioxane in methylene chloride and sodium hydroxide. After BOA was sulfonated, the BOS—NA salt was isolated as the sodium salt by evaporating the solvent mixture.

The major disadvantages of this prior art process are that the preparation of zonisamide through the BOS—Na intermediate not only imparts color to zonisamide but also results in additional steps such as isolation and drying.

Accordingly, there remains a need for an improved process for preparing zonisamide that eliminates and/or reduces the problems of the prior art on a commercial scale in a convenient and cost efficient manner.

### SUMMARY OF THE INVENTION

One aspect of the present invention is to prepare 1,2-benzisoxazole-3-acetic acid (BOA) without the use of metallic sodium, and thus the process of this invention is substantially less hazardous.

Another aspect of the present invention is to prevent the formation of side-products, e.g., oximes, and therefore significantly increasing the yield of BOA. By preventing the formation of side-products such as oximes, the burden of removing the oxime side-product with ether, which by itself is hazardous, is substantially reduced.

Another aspect of the present invention is to prepare zonisamide and derivatives thereof by eliminating the step of forming the salt of benzisoxazole methane sulfonic acid thereby reducing additional steps such as isolation and drying.

Another aspect of the present invention is to prepare zonisamide and derivatives thereof by avoiding the step of isolation of benzisoxazole methane sulfonic acid (BOS).

Another aspect of the present invention is to prepare zonisamide and derivatives thereof by avoiding the step of isolation of BOS—CL.

Another aspect of the present invention is to prepare zonisamide and derivatives thereof by forming BOS and BOS—Cl in-situ, i.e., in a single pot reaction, using the starting material BOA.

Accordingly, in one embodiment of the present invention, an improved process for the preparation of zonisamide or a derivative thereof is provided comprising:

(a) reacting 1,2-benzisoxazole-3-methane-sulfonic acid with a halogenating agent in a first organic solvent to provide benzisoxazole methane sulfonyl halide; and,

(b) reacting benzisoxazole methane sulfonyl halide with an amine in a second organic solvent to form zonisamide or a derivative thereof.

In accordance with another embodiment of the present invention, an improved process for the preparation of zonisamide or a derivative thereof is provided comprising:

(a) reacting 1,2-benzisoxazole-3-acetic acid with chlorosulfonic acid and dioxane in a halogenated hydrocarbon solvent to produce 1,2-benzisoxazole-3-methane sulfonic acid;

(b) reacting 1,2-benzisoxazole-3-methane sulfonic acid with a halogenating agent in a first organic solvent to provide benzisoxazole methane sulfonyl halide; and,

(c) reacting benzisoxazole methane sulfonyl chloride with an amine in a second organic solvent to form zonisamide or a derivative thereof. If desired, the step of forming 1,2-benzisoxazole-3-methane sulfonic acid can further include impregnating the 1,2-benzisoxazole-3-acetic acid with an inorganic salt such as, for example, NaCl and KCl.

The advantages of the processes of the present invention include:

1. By converting 1,2-benzisoxazole-3-acetic acid into 1,2-benzisoxazole-3-methane sulfonic acid instead of the salt of the 1,2-benzisoxazole-3-methane-sulfonic acid, additional steps such as isolation and drying are avoided. By avoiding the additional steps as required by the prior art, the overall reaction time is substantially reduced in forming the end product zonisamide or a derivative thereof. Also, by avoiding the step of converting 1,2-benzisoxazole-3-acetic acid into the sodium salt of 1,2-benzisoxazole-3-methane sulfonic acid, an improved appearance of the end product zonisamide is achieved.

2. By impregnating 1,2-benzisoxazole-3-acetic acid with an inorganic salt during the conversion step of 1,2-benzisoxazole-3-acetic acid into 1,2-benzisoxazole-3-methane sulfonic acid allows for the sulfonic acid to be formed as a generally fine powder thereby making the sulfonic acid a better reactive species.

3. The use of POCl₃ as a chlorinating agent in a toluene medium provides improved yields of BOS—Cl as co-distillation of POCl₃ with toluene at atmospheric pressure allows for less decomposition of BOS—Cl.

### DETAILED DESCRIPTION OF THE PREFERRED EMBODIMENTS

The following abbreviations are used herein: 1,2-benzisoxazole-3-acetic acid (BOA); 1,2-benzisoxazole-3-methane sulfonic acid (BOS); sodium salt of 1,2-benzisoxazole-3-methane sulfonic acid (BOS—Na); and benzisoxazole methane sulfonyl chloride (BOS—Cl).

The present invention provides improved processes for the preparation of zonisamide or derivatives thereof. In one embodiment, the process includes at least (a) reacting BOS with a halogenating agent in a first organic solvent to provide benzisoxazole methane sulfonyl halide; and, (b) reacting the benzisoxazole methane sulfonyl halide with an amine in a second organic solvent to form zonisamide or a derivative thereof.

BOS can be prepared by, for example, reacting BOA with chlorosulfonic acid in a halogenated hydrocarbon solvent. Generally, the reaction can be carried out by (a) preparing a mixture of the chlorosulfonic acid and halogenated hydrocarbon solvent and optionally dioxane, (b) adding BOA to the mixture, and (c) heating the mixture.

Suitable halogenated hydrocarbon solvent include, but are not limited to, methylene chloride, ethylene dichloride (EDC), chloroform, carbon tetrachloride and chlorobenzene ortho dichlorobenzene and the like and mixtures thereof.

The reaction can be carried out at a temperature of about −5° C. to about 80° C. and preferably from about 50° C. to about 60° C. The concentration of the halogenated hydrocarbon solvent will ordinarily range from about 2 to about 10 v/w of the BOA. The BOA can be added to the mixture in a molar ratio of BOA:chlorosulfonic acid of about 1:1 to about 1:2 and more preferably about 1:1 to about 1:1.1 in one portion.

If desired, BOA can first be impregnated with an inorganic salt, e.g., sodium chloride, potassium chloride, and magnesium chloride, after azeotropic removal of water. This advantageously may allow for the resulting BOS obtained to be formed as a generally fine powder thereby making it a better reactive species for subsequent steps. Generally the amount of inorganic salt used can range from 0.25 to about 0.75 weight percent, based on the weight of BOA.

Next, BOS can be reacted with a halogenating agent in a first organic solvent to provide benzisoxazole methane sulfonyl halide which is thereafter converted to zonisamide or a derivative thereof. Suitable halogenating agents include, but are not limited to, SOCl₂, POCl₃, PCl₅, S₂Cl₂, PBr₃, PCl₃ and the like and mixtures thereof. A preferred halogenating agent for use herein is POCl₃.

The first organic solvent can include but is not limited to, aliphatic organic solvents, aromatic organic solvents and the like and mixtures thereof. Examples of aliphatic solvents include pentane, hexane and the like and mixtures thereof. Examples aromatic solvents include benzene, toluene, xylene and the like and mixtures thereof. Aromatic solvents are preferred with toluene being most preferred.

The temperature of the reaction between the BOS and halogenating agent may range from about 90° C. to about 150° C. The halogenating agent will ordinarily be added to the BOS in an amount ranging from 1 to about 10 molar equivalents per equivalent of the BOS.

The reaction of BOS with a halogenating agent in an organic solvent, e.g., chlorinating BOS with POCl₃ in a toluene medium to provide BOS—Cl, advantageously may result in higher yields of the resulting intermediate such as BOS—Cl, e.g., greater than about 90% and preferably greater than about 95%, than that of the prior art. Also, the co-distillation of POCl₃ with toluene at atmospheric pressure can account for less decomposition of the BOS—Cl.

After the reaction of BOS with a halogenating agent, any excess halogenating agent may be distilled off. The benzisoxazole methane sulfonyl halide may then be thereafter converted to zonisamide or a derivative thereof by reacting benzisoxazole methane sulfonyl chloride with an amine in a second organic solvent. For example, the benzisoxazole methane sulfonyl halide can be initially quenched in a second organic solvent and then saturated with a suitable amine. The second organic solvent includes, but is not limited to, acetates such as, methyl acetate, ethyl acetate, propyl acetate, isopropyl acetate, n-butyl acetate, isobutyl acetate, and the like and mixtures thereof. Useful amines can be those of the formula R₁NH₂ wherein R₁ is hydrogen or an alkyl of 1 to about 4 carbons, e.g., ammonia. Generally, the reaction can be carried out using any appropriate amine source, preferably ammonia gas (R₁═H) from an ammonia gas generating source, to provide the zonisamide or a derivative thereof. This avoids problems of forming a clumpy mass when ammonia gas directly purges the reaction mass. The zonisamide or derivative thereof obtained herein is of relatively high purity, e.g., greater than about 90%, preferably greater than about 98% and more preferably greater than about 99%.

In another embodiment, the process includes at least (a) reacting BOA with chlorosulfonic acid and dioxane in a halogenated hydrocarbon solvent to produce BOS; (b) reacting BOS with a halogenating agent in the presence of an organic solvent, e.g., toluene, to provide benzisoxazole methane sulfonyl halide; and, (c) reacting benzisoxazole methane sulfonyl halide with an amine of the formula R₁NH₂ wherein R₁ is hydrogen or an alkyl of 1 to about 4 carbons, in ethyl acetate to form zonisamide.

In a preferred embodiment of the present invention, zonisamide (I) can be prepared as shown in Scheme II: 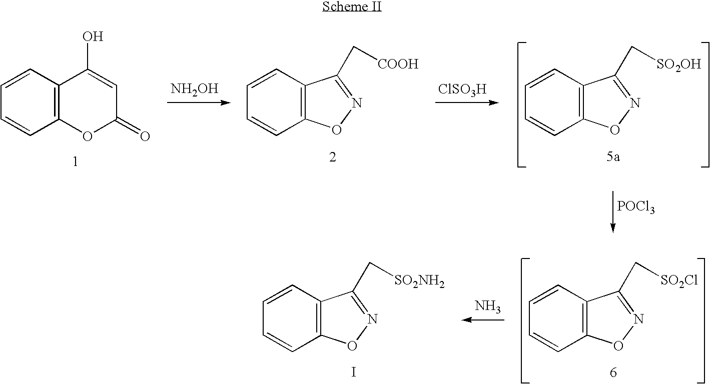
As shown in Scheme II, a Posner reaction of 4-hydroxy coumarin (1) with hydroxylamine in an alcohol solvent is carried out to form BOA (2). Sodium acetate is used in this step to quench the HCl. Suitable alcohols include the lower alcohol, e.g., methanol, ethanol, n-butanol, iso-propyl-alcohol, iso-butanol, amyl-alcohol, iso-amyl-alcohol and the like and mixtures thereof. The second step is chlorosulfonation of BOA (2) with chlorosulfonic acid in an EDC/Dioxane mixture to get in-situ 1,2-benzisoxazole-3-methane sulfonic acid [BOS (5a)]. The BOS (5a) is chlorinated using POCl₃ in the presence of an organic solvent to provide BOS—Cl (6). The amidation of BOS—Cl (6) is then carried out using, for example, ammonia gas, in ethyl acetate to provide the product zonisamide (I).

The following example is provided to enable one skilled in the art to practice the invention and are merely illustrative of the invention. The example should not be read as limiting the scope of the invention as defined in the claims.

### EXAMPLE

Step (1) Preparation of 1,2-benzisoxazol-3yl-acetic acid.

Hydroxyl amine hydrochloride (750.0 g, 10.80 mol) was added to a stirred solution of 4-Hydroxy coumarin (500 g, 3.086 mol) in methanol (5.0 liters) at 25-30° C. Sodium acetate (885.0 g, 10.80 mol) was added to the above solution lot wise in half an hour. The reaction mass was stirred at 25-30° C. for half an hour, heated to reflux (65-70° C.) and maintained at reflux for 5-6 hours. After completion of the reaction (by TLC), methanol was distilled under vacuum (<50° C.). After complete removal of methanol, 7.0 liters of water was added to the residue and the resulting solution was cooled to 10-15° C. The pH of the reaction mass was adjusted to 2-3 with 50% HCl and stirred the reaction for one hour at 10-15° C. The solid obtained was filtered and washed with 2.0 lit of water. The solid was dried at 55-60° C. till LOD reached <1.0%; N. Wt 410.0 g., Yield 62%, Purity 99% by HPLC.

Step (2) Preparation of 1,2-benzisoxazole-3-methane sulfonic acid.

Chlorosulfonic acid (364 g., 3.12 mol) was added slowly under stirring to cooled solution of 1,2-dichloroethane (1600.0 ml) at a temperature of 0-5° C. To the above solution, dioxane (274.0 g, 3.12 mol) was added dropwise over a period of 30-40 minutes. The reaction mass was stirred for half an hour at a temperature of 0-5° C. and 1,2-benzisoxazol-3-yl acetic acid (500.0 g, 2.82 moles) was added in portions over half an hour. After completion of the addition, the reaction mass was heated slowly to 10-15° C. and maintained at the same temperature for half an hour. The reaction temperature was further raised to 30-35° C. in half an hour and maintained for 3.0 hrs. The reaction mass was heated to 55-60° C. and maintained for 4-5 hours. After completion of the reaction (by TLC) the reaction mass was cooled to 10-15° C. and water (2.0 liters) was added to get a clear solution, which was stirred for half an hour. The layers were separated and the aqueous layer was washed with ethyl acetate (1.0 liters). The aqueous layer was cooled distilled atmospherically to half volume. The residue was cooled to 25-30° C. and 250 gm sodium chloride was added and stirred for 30 minutes. Toluene (5 liters) was added and the reaction mixture was heated to reflux. Water was distilled azeotropically until the moisture content reached 0.2%.

Step (3) Preparation of Zonisamide

To a stirred suspension of 1,2-benzisoxazole-3-methane sulfonic acid (600.0 g, 2.08 mol) in toluene (5 liters) was added phosphorous oxychloride (1200.0 ml, 12.5 mol). The reaction mass was heated to reflux (100-105° C.) in half an hour and maintained for 2.0 hrs. After completion of the reaction (by TLC) excess phosphorous oxychloride and toluene were distilled off at atmospheric pressure. The traces of the reagents were removed by further distilling with toluene (2.4 liters) and ethyl acetate (4.8 lt). The residue obtained after the distillation was cooled to 25-30° C. and diluted with 2.4 of ethyl acetate and quenched with a solution of ethyl acetate (15.0 liters) saturated with anhydrous ammonia gas at 0-20° C. over a period of 2-3 hrs. After quenching, anhydrous ammonia gas was purged to the reaction mass below 25° C., until pH reaches 9-10 and stir for 12 hrs at pH 9-10. The reaction mass was diluted with 4.8 liters water. The layers were separated and the organic layer was extracted with 20 liters ethyl acetate. The organic layers were combined and treated with charcoal at 70-75° C. The hot solution was filtered and distilled to 1.5-2.0 v/w. The organic layer was cooled to 5-10° C. and maintained for 1.0 hr. The solid obtained was filtered and washed with prechilled ethyl acetate (500.0 ml) and dried at 60-65° C. till LOD reached <0.5%; N. Wt 450.0 g, yield 76%, purity 99.95%. The product obtained passes all regulatory requirements. The spectral data such as IR, ¹H-NMR, ¹³C-NMR, Mass are consistent with the proposed structure.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. A process for the preparation of zonisamide or a derivative thereof comprising:
(a) reacting 1,2-benzisoxazole-3-methane-sulfonic acid (BOS) with a halogenating agent in a first organic solvent to provide benzisoxazole methane sulfonyl halide; and
(b) reacting benzisoxazole methane sulfonyl halide with an amine in a second organic solvent to form zonisamide or a derivative thereof.

2. The process of claim 1, wherein the halogenating agent is selected from the group consisting of SOCl₂, POCl₃, PCl₅, S₂Cl₂, PBr₃, PCl₃ and mixtures thereof.

3. The process of claim 1, wherein the first organic solvent is selected from the group consisting of an aliphatic solvent, an aromatic solvent and mixtures thereof.

4. The process of claim 3, wherein the aliphatic solvent is selected from the group consisting of pentane, hexane and mixtures thereof.

5. The process of claim 3, wherein the aromatic solvent is selected from the group consisting of benzene, toluene, xylene and mixtures thereof.

6. The process of claim 1, wherein the halogenating agent is POCl₃ and the first organic solvent is toluene.

7. The process of claim 1, wherein the temperature of the reaction between BOS and the halogenating agent is about 90° C. to about 150° C.

8. The process of claim 1, wherein the second organic solvent is selected from the group consisting of methyl acetate, ethyl acetate, propyl acetate, isopropyl acetate, n-butyl acetate, isobutyl acetate and mixtures thereof.

9. The process of claim 1, wherein the amine is of the formula R₁NH₂ wherein R₁ is hydrogen or an alkyl of 1 to about 4 carbons.

10. The process of claim 1, wherein the second organic solvent is ethyl acetate and the amine is ammonia.

11. The process of claim 10, wherein the ammonia is ammonia gas.

12. A process for the preparation of zonisamide or a derivative thereof comprising:
(a) reacting 1,2-benzisoxazole-3-acetic acid (BOA) with chlorosulfonic acid and dioxane in a halogenated hydrocarbon solvent to produce BOS;
(b) reacting BOS with a halogenating agent in a first organic solvent to provide benzisoxazole methane sulfonyl halide; and
(c) reacting benzisoxazole methane sulfonyl halide with an amine in a second organic solvent to provide zonisamide or a derivative thereof.

13. The process of claim 12, further comprising prior to step (a) reacting hydroxycoumarin with hydroxylamine in the presence of sodium acetate in an alcohol solvent to produce BOA.

14. The process of claim 12, wherein step (a) comprises (i) preparing a mixture of chlorosulfonic acid, the halogenated hydrocarbon solvent and dioxane; (ii) adding BOA to the mixture; and (iii) heating the mixture.

15. The process of claim 14, wherein the halogenated hydrocarbon solvent is dichloroethane.

16. The process of claim 12, wherein the halogenated hydrocarbon solvent is selected from the group consisting of methylene chloride, ethylene dichloride and mixtures thereof.

17. The process of claim 12, further comprising impregnating BOA with an inorganic salt.

18. The process of claim 17, wherein the inorganic salt is selected from the group consisting of NaCl, KCl, MgCl and mixtures thereof.

19. The process of claim 12, wherein the halogenating agent is selected from the group consisting of SOCl₂, POCl₃, PCl₅, S₂Cl₂, PBr₃, PCl₃ and mixtures thereof.

20. The process of claim 12, wherein the first organic solvent is selected from the group consisting of an aliphatic solvent, an aromatic solvent and mixtures thereof.

21. The process of claim 12, wherein the halogenating agent is POCl₃ and the first organic solvent is toluene.

22. The process of claim 9, wherein the temperature of the reaction between BOS and the halogenating agent is about 90° C. to about 150° C.

23. The process of claim 1, wherein the second organic solvent is selected from the group consisting of methyl acetate, ethyl acetate, propyl acetate, isopropyl acetate, n-butyl acetate, isobutyl acetate and mixtures thereof.

24. The process of claim 12, wherein the amine is of the formula R₁NH₂ wherein R₁ is hydrogen or an alkyl of 1 to about 4 carbons.

25. The process of claim 12, wherein the second organic solvent is ethyl acetate and the amine is ammonia.

26. The process of claim 25, wherein the ammonia is ammonia gas.

27. The process of claim 21, wherein the second organic solvent is ethyl acetate and the amine is ammonia.

28. The process of claim 12, further comprising the step of purifying zonisamide.

29. Zonisamide having a purity equal to or greater than about 90% prepared in accordance with the process of claim 1.

30. Zonisamide having a purity equal to or greater than about 90% prepared in accordance with the process of claim 12.

31. Zonisamide prepared in accordance with the process of claim 1.

32. Zonisamide prepared in accordance with the process of claim 12.
